(19) United States
(12) Patent Application Publication
(10) Pub. No.: US 2023/0290449 A1
(43) Pub. Date: Sep. 14, 2023

(54) SYSTEM AND METHOD FOR BIOLOGIC TESTING

(71) Applicant: HuddleNurse, PLLC, Lubbock, TX (US)

(72) Inventors: Michelle Coffey, Wolfforth, TX (US); Shannon Turnbow, Lubbock, TX (US); Trey Kessee, Lubbock, TX (US); Shanda Brown, Austin, TX (US)

(73) Assignee: HuddleNurse, PLLC, Lubbock, TX (US)

(21) Appl. No.: 18/181,121

(22) Filed: Mar. 9, 2023

Related U.S. Application Data
(60) Provisional application No. 63/318,654, filed on Mar. 10, 2022.

(51) Int. Cl. G16H 10/40

(52) U.S. Cl. CPC G16H 10/40

(57) ABSTRACT

A biologic test system (BTS) includes a kiosk having a user interface, a dispensing system, and a locker. The locker can be configured to remain locked for a predetermined period of time. A method of conducting a biologic test includes providing a kiosk, providing a medical professional computer system, and dispensing a biologic test kit from the kiosk.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

In the field of biologic testing, such as, but not limited to, testing biological samples for the COVID-19 virus, test kits exist that provide all require component parts, chemicals, and biologics necessary to rapidly conduct a test. Some of the more accurate test kits are normally provided to and administered by medical professionals in a traditional or formal medical services setting, such as a physician's office or hospital. However, there is a need for improvements to accessibility test kits and medical professional assistance.

### BRIEF DESCRIPTION OF THE DRAWINGS

FIG. 1 is a schematic view of a biologic testing system according to an embodiment of this disclosure.

FIG. 2 is an oblique view of a kiosk according to an embodiment of this disclosure.

FIG. 3 is another oblique view of the kiosk of FIG. 2.

FIG. 4 is a front view of the kiosk of FIG. 2.

FIG. 5 is a rear view of the kiosk of FIG. 2.

FIG. 6 is a left view of the kiosk of FIG. 2.

FIG. 7 is a right view of the kiosk of FIG. 2.

FIG. 8 is a top view of the kiosk of FIG. 2.

FIG. 9 is a bottom view of the kiosk of FIG. 2.

FIG. 10 is a partial oblique view of the kiosk of FIG. 2.

FIG. 11 is another partial oblique view of the kiosk of FIG. 2.

FIG. 12 is a partial top view of the kiosk of FIG. 2.

FIG. 13 is a partial left view of the kiosk of FIG. 2.

FIG. 14 is a partial oblique view of the kiosk of FIG. 2.

FIG. 15 is a another partial oblique view of the kiosk of FIG. 2.

FIG. 16 is a flowchart of a method of operating a biologic testing system according to an embodiment of this disclosure.

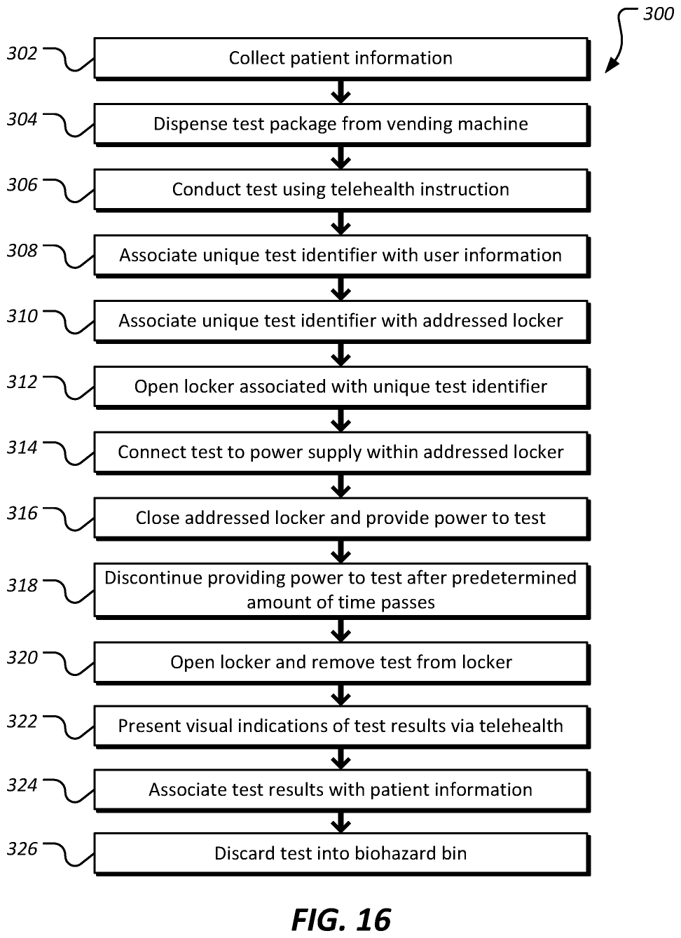

### DETAILED DESCRIPTION

In this disclosure, reference may be made to the spatial relationships between various components and to the spatial orientation of various aspects of components as the devices are depicted in the attached drawings. However, as will be recognized by those skilled in the art after a complete reading of this disclosure, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

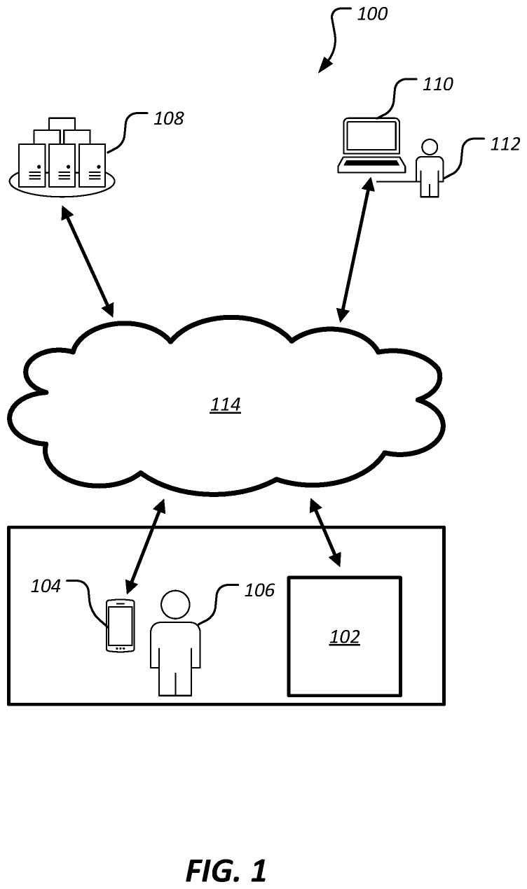

Referring to FIG. 1, a schematic diagram of a biologic testing system (BTS) 100 is shown. BTS 100 comprises a kiosk 102, a patient smartphone 104 for use by a patient 106, a data system 108, and a medical professional computer system 110 for use by a medical professional 112. One or more of the kiosk 102, patient smartphone, data system 108, and medical professional computer system 110 can communicate with each other via a computer network 114, such as the internet. BTS 100 is utilized to supply a patient 106 with both a biologic test kit and medical professional support for conducting the biologic test. Generally, kiosk 102 can be stocked with test kits to be vended to patients, can accept payment for test kits, can provide a space for storing and powering test kits, and can receive test kit waste. The patient smartphone 104 and medical professional computer system 110 can be used for telehealth support services toward the end of conducting the test and the data system 108 can be used to temporarily or permanently store or manipulate data associated with the patient, test kit, and/or testing results.

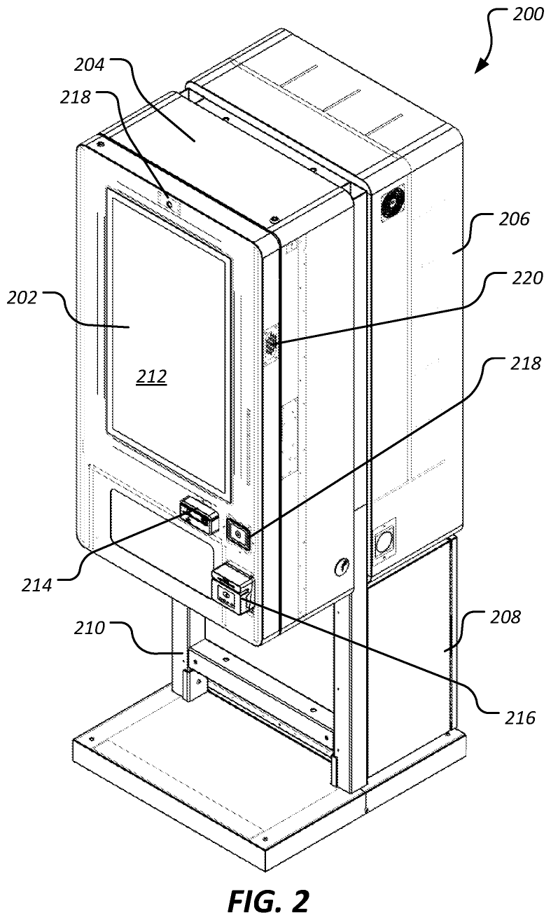

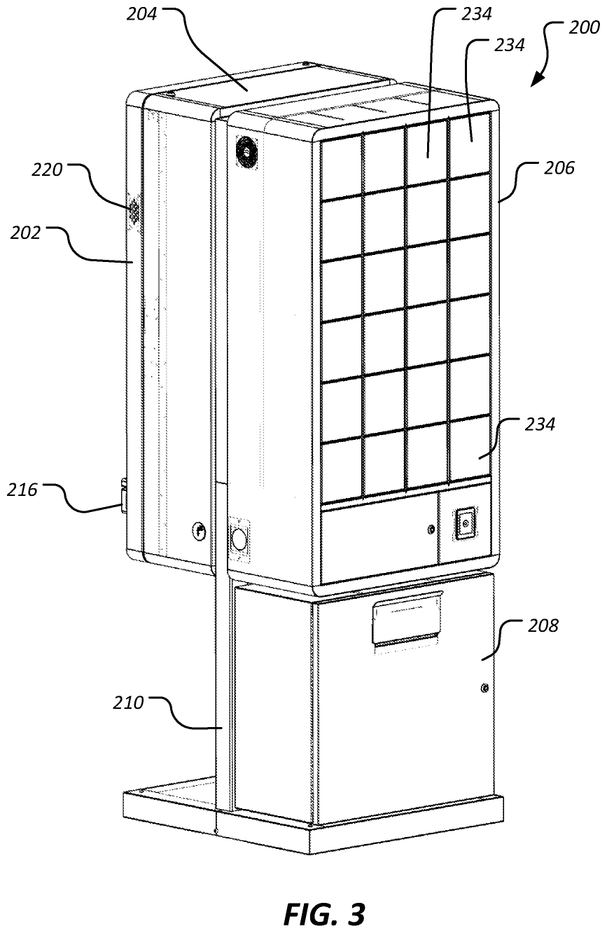

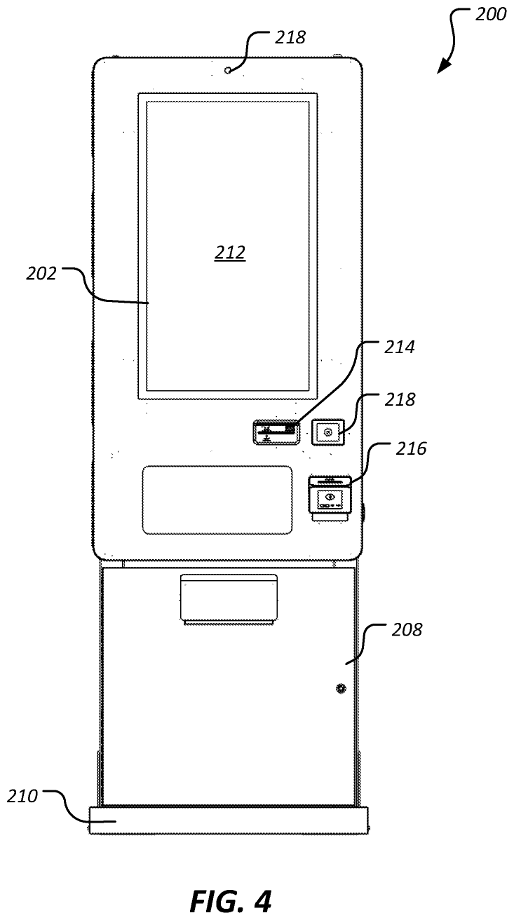

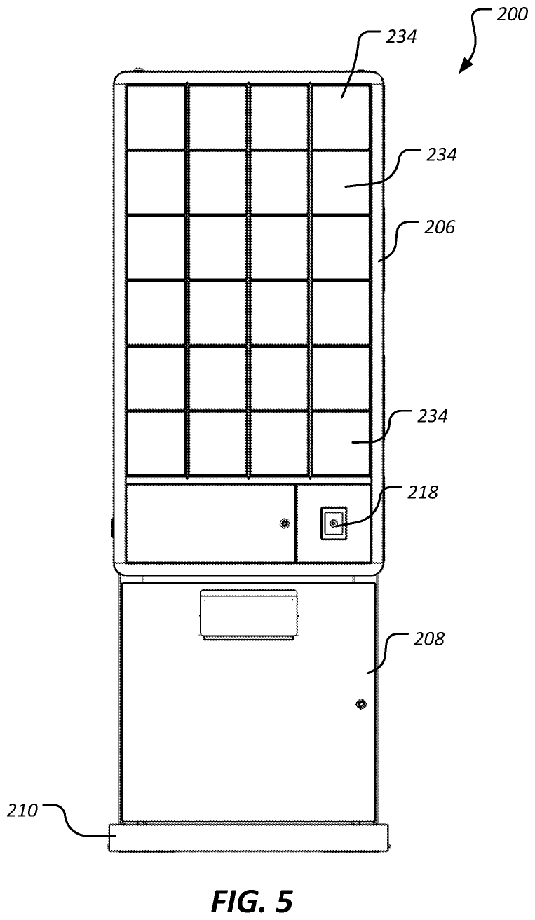

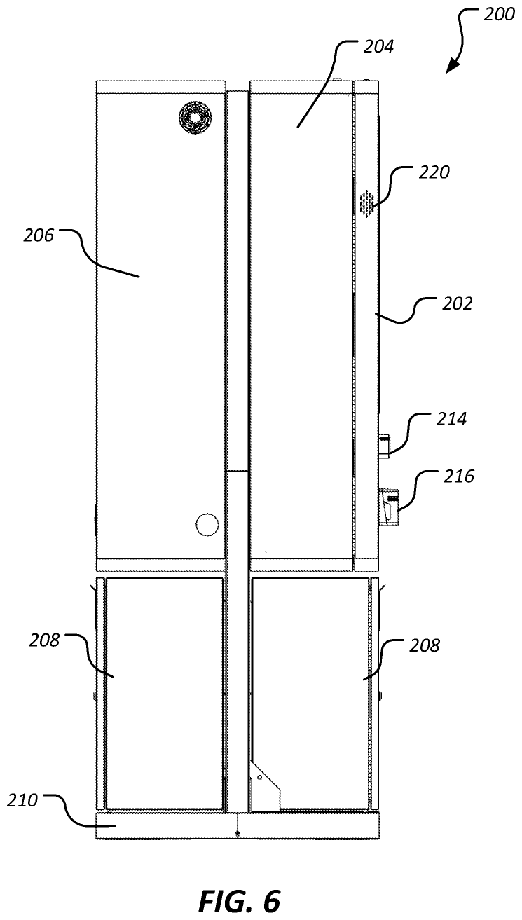

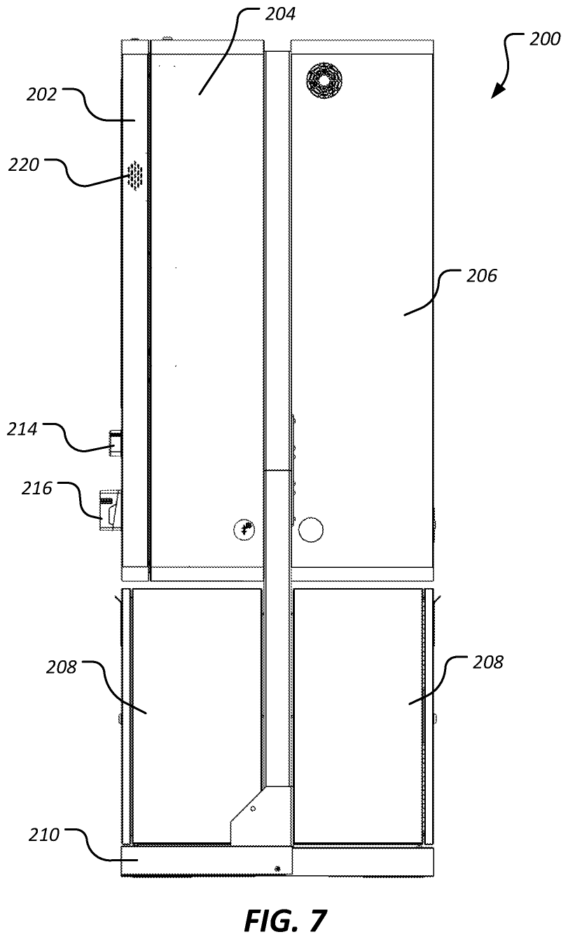

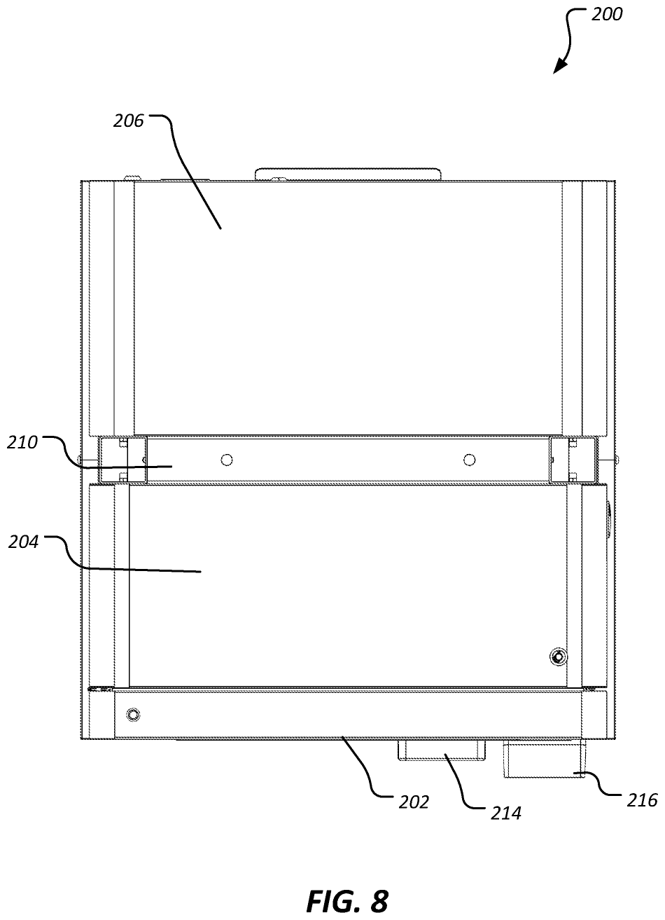

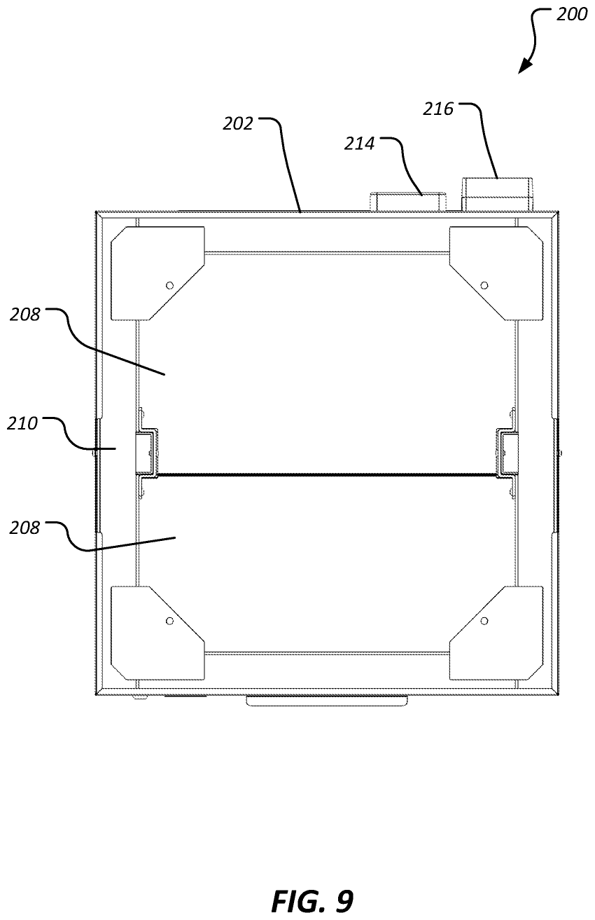

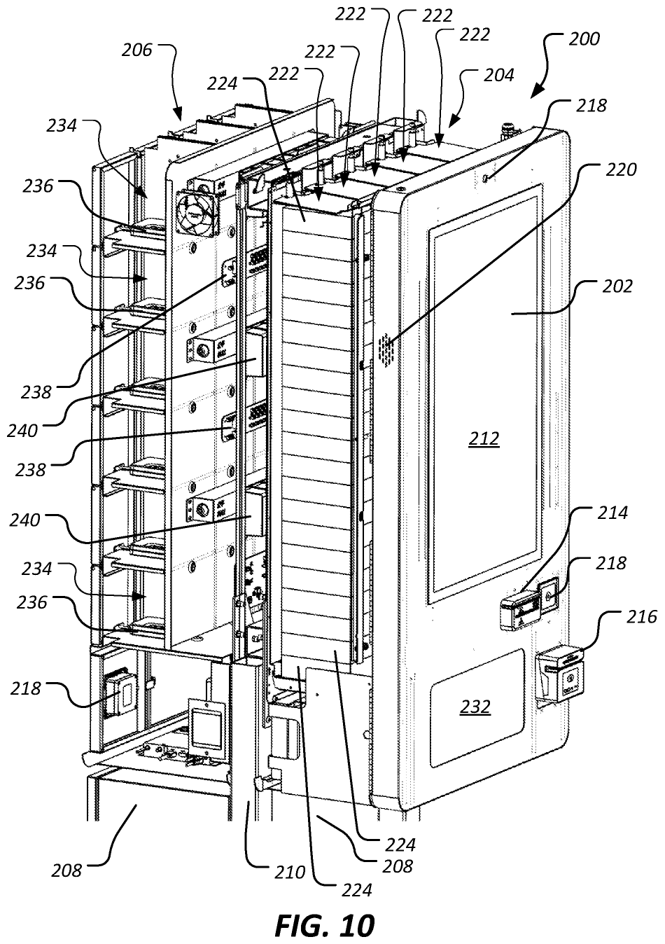

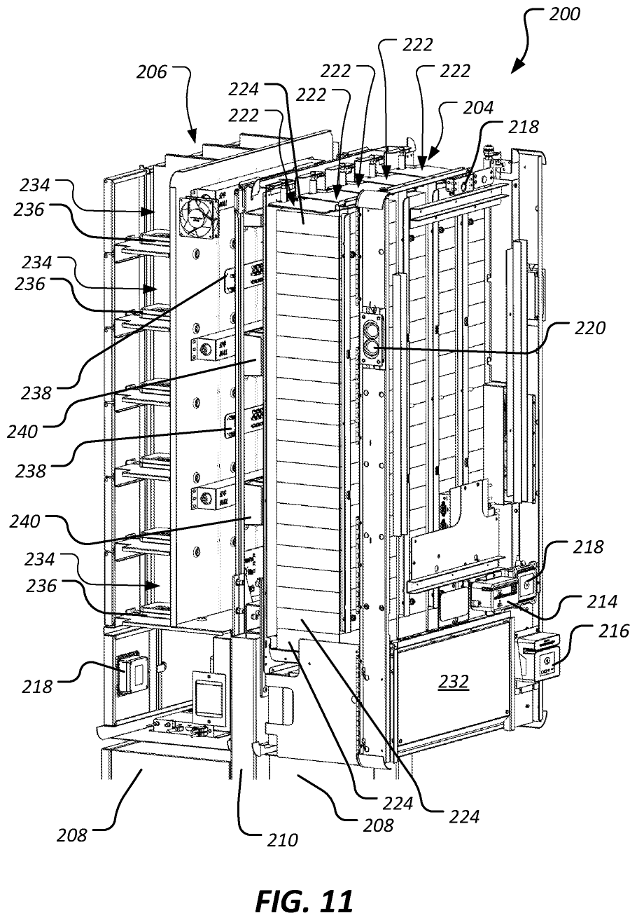

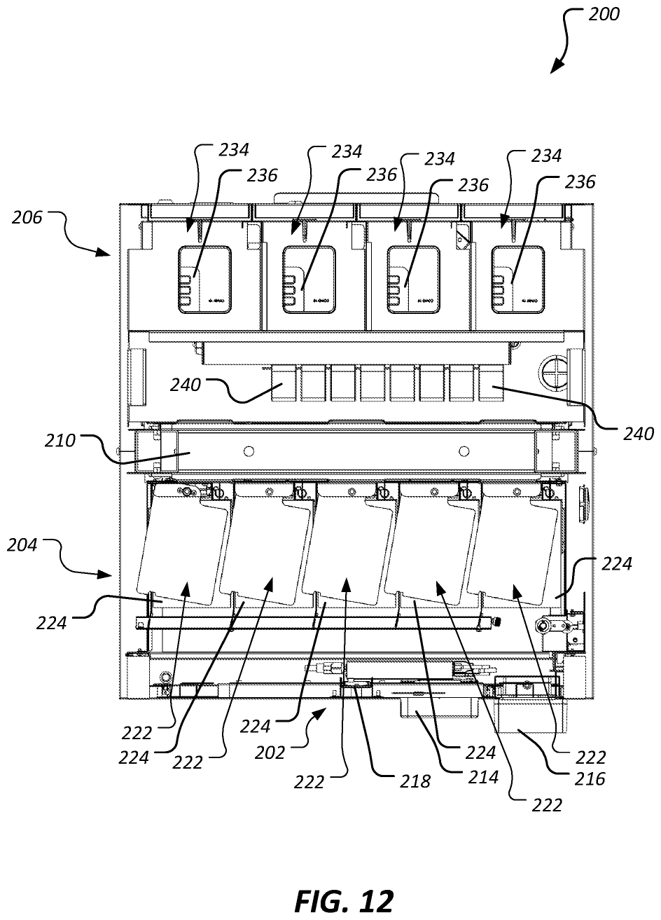

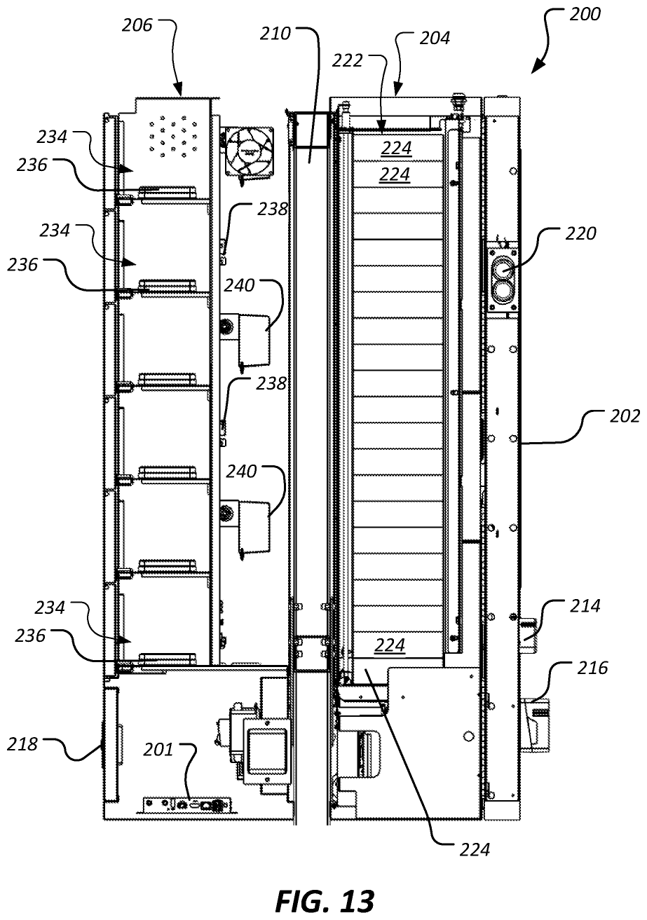

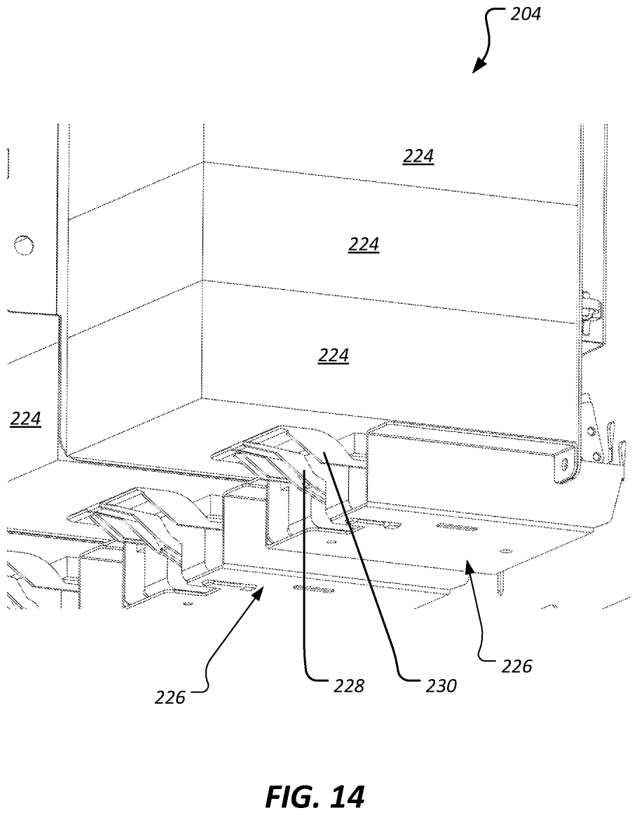

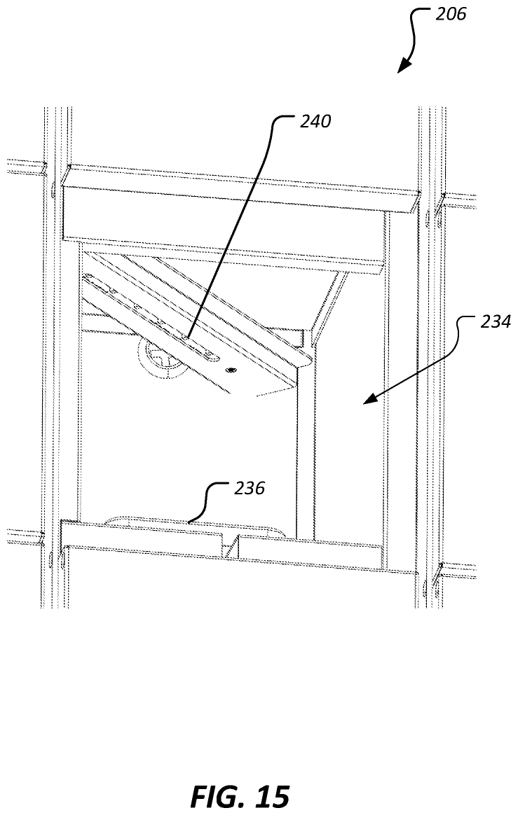

Referring now to FIGS. 2-15, a kiosk 200 is shown. Kiosk 200 is substantially similar to kiosk 102 insofar as it can be utilized in BTS 100. Most generally, kiosk 200 comprises a user interface 202, a vending system 204, a locker system 206, a biohazard trash bin 208, and a frame 210. User interface 202 comprises a specially programmed general purpose computer 201 for accepting patient information and providing information to patients via a touch screen interface 212. User interface 202 further comprises a special purpose magnetic card reader 214, a payment reader 216, an optical sensors 218, and a speaker 220.

Vending system 204 generally comprises a plurality of segregated storage columns 222 configured to receive and selectively retain vertically stacked test kits 224. Each column 222 is associated with a separate drop system 226 configured to selectively move a drop actuator 228 that allows rotation of a drop arm 230 from a vertically supportive position to an unsupportive position that allows a bottom located test kit to fall from the stack and become retrievable via the dispenser door 232.

Locker system 206 generally comprises a plurality of segregated lockers 234, each being configured to receive a test 236 of a test kit 224 therein. Each of the lockers 234 is separately electronically addressable and separately electromechanically lockable, the locking and unlocking being controlled by control boards 238. Further, each locker 234 is provided with a power source, in this embodiment, in the form of a plug (not shown) supplying direct current power from a direct current power supply 240. In cases where tests 236 require being powered to effectively process the test, such as some conventional Polymerase Chain Reaction (PCR) rapid tests, control boards 238 can utilize information about opening and closing of lockers 234 and/or plugging in of a test 236 to control a timer for controlling locking of the locker.

In operation, of a BTS substantially similar to BTS 100 that comprises a kiosk substantially similar to kiosk 200, a method 300 may begin at block 302 by operating the kiosk to collect patient information. Additionally, payment information can be gathered by the kiosk. At block 304, once the patient has entered the patient information and paid for a test kit, the dispensing system of the kiosk can dispense a test kit. Next, a patient can use a smartphone or similarly capable device to received telehealth support from a medical professional who can use a medical provisional computer system to videoconference with the patient. The medical professional can visually and verbally guide the patient through the test sample preparation procedure and otherwise assist the patient with preparing the test for execution. Next, at block 308, the kiosk can associate a unique test identifier with the user information, in some cases by using optical sensors to scan a QR code of the test kit to provide a unique test identifier. Next, at block 310, the unique test identifier and/or the patient information can be associated and further as sociated with a locker to address the locker to the patient and/or test. The method can continue at block 312 by unlocking and/or automatically opening the addressed locker. Next at 314, the patient can place the prepared test into the addressed locker, connect the test to the power supply plug. Next at 316, the locker can be closed to start providing power to the test and also to start a countdown timer configured to supply power to the test for a prescribed period of time. Next at block 318, the kiosk can stop providing power to the test after the predetermined period of time has passed and the test has fully processed. Next at block 320, the locker can be opened and the test can be removed from the locker. The method 300 can continue at block 322 by the patient using the smartphone to again connect to a medical professional and by presenting visual indication of the test results to the medical professional, namely, by directing a camera of the smartphone at the visual indicators of the completed test. Next at block 324, the kiosk, the smartphone, the medical professional computer, and/or the data system 108 can associate the test results as confirmed by the medical professional with the patient information to generate a medical professional endorsed test result health record for the patient. Finally at block 326, the patient can discard all test kit contents into a biohazard bin of the kiosk.

In some embodiments, a locker 234 can comprise an ultraviolet light source 242 configured to sanitize the interior of the locker 234 after a completed test 236 has been removed from the locker 234. In some cases, the kiosk 200 can maintain the locker 234 in a locked configuration for during a mandatory cleaning cycle that can automatically occur upon closing the locker 234 after removal of the completed test 236.

At least one embodiment is disclosed, and variations, combinations, and/or modifications of the embodiment(s) and/or features of the embodiment(s) made by a person having ordinary skill in the art are within the scope of this disclosure. Alternative embodiments that result from combining, integrating, and/or omitting features of the embodiment(s) are also within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₛ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 95 percent, 98 percent, 99 percent, or 100 percent. Moreover, any numerical range defined by two R numbers as defined in the above is also specifically disclosed.

Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention. Also, the phrases “at least one of A, B, and C” and “A and/or B and/or C” should each be interpreted to include only A, only B, only C, or any combination of A, B, and C.

What is claimed is:

1. A biologic test system (BTS), comprising:
a kiosk, comprising:
a user interface;
a dispensing system; and
a locker.

2. The BTS of claim 1, wherein the locker is configured to remain locked for a predetermined period of time.

3. The BTS of claim 1, wherein the locker comprises a power source.

4. The BTS of claim 1, wherein the locker comprises a disinfecting light.

5. The BTS of claim 1, wherein the dispensing system is configured to accommodate a vertical stack of test kits and wherein the dispensing system is configured to dispense a lowest located test kit within the vertical stack of test kits.

6. The BTS of claim 1, wherein the dispensing system comprises an actuator arm configured to move to allow dispensing of a test kit.

7. The BTS of claim 1, wherein the kiosk comprises a plurality of lockers and a plurality of power supplies associated with the plurality of lockers.

8. The BTS of claim 1, wherein at least one of the plurality of lockers is addressable to be associated with patient information entered via the user interface.

9. The BTS of claim 1, further comprising:
a frame that vertically supports the user interface, the dispensing system, and the locker, wherein the user interface and the dispensing system face forward while the locker faces backward.

10. The BTS of claim 9, further comprising:
a biohazard trash bin carried by the frame and disposed below at least one of the dispensing system and the locker.

11. A method of conducting a biologic test, comprising:
providing a kiosk;
providing a medical professional computer system;
dispensing a biologic test kit from the kiosk.

12. The method of claim 11, further comprising:
operating the kiosk to receive payment for the biologic test prior to dispensing the biologic test.

13. The method of claim 11, further comprising:
operating the kiosk to receive patient information.

14. The method of claim 11, further comprising:
operating the medical professional computer system to provide telehealth services.

15. The method of claim 11, further comprising:
opening a locker of the kiosk to receive the test kit.

16. The method of claim 15, further comprising:
connecting the test kit to a power supply of the locker.

17. The method of claim 16, further comprising:
locking the locker for a predetermined period of time.

18. The method of claim 17, further comprising:
transmitting visual information about the test kit to the medical professional computer system.

19. The method of claim 18, further comprising:
receiving test result information from the medical professional computer system.

20. The method of claim 19, further comprising:
associating the test result information with patient information.
